(19) United States
(12) Patent Application Publication
(10) Pub. No.: US 2025/0111505 A1
(43) Pub. Date: Apr. 3, 2025

(54) ORGANOID SELECTION DEVICE AND METHOD

(71) Applicant: THE CATHOLIC UNICERSITY OF KOREA INDUSTRY-ACADEMIC COOPERATION FOUNDATION, Seoul (KR)

(72) Inventors: Do Hyun KIM, Seoul (KR); Seungchul LEE, Pohang-si (KR); Sung Won KIM, Seoul (KR); Mi Hyun LIM, Siheung-si (KR); Keon Hyeok PARK, Daejeon (KR); Seung Min SHIN, Ulsan (KR)

(21) Appl. No.: 18/832,597

(22) PCT Filed: Jan. 17, 2023
(86) PCT No.: PCT/KR2023/000815

§ 371 Date: Jul. 24, 2024

(30) Foreign Application Priority Data
Feb. 21, 2022 (KR) 10-2022-0022545

(51) Int. Cl. G06T 7/00; G06T 7/11

(52) U.S. Cl. CPC G06T 7/0012; G06T 7/11; G06T 2207/10056; G06T 2207/20081; G06T 2207/20084; G06T 2207/30024

(57) ABSTRACT

The present disclosure relates to an organoid selection device and method, and, particularly, can provide an organoid selection device and method, which enable a mature organoid to be selected from an image by using an artificial intelligence model. Particularly, provided is a device, which constructs and verifies an artificial intelligence model so as to use only image information about an organoid, and thus can more accurately select a mature organoid exhibiting uniform efficacy.

### TECHNICAL FIELD

The present embodiments provide an organoid selection device and method.

### BACKGROUND ART

Various attempts are being made to apply image analysis technology using artificial neural networks to images and pathological tissue images in the medical field as such image analysis technology advances. In particular, analysis methods using artificial intelligence for recognizing patterns from images and performing learning and selection are being applied to various medical fields including radiology. Further, ‘mini organs’ and organoids have recently attracted attention, and these organoids may be used to reveal crucial parts of tissue generation, homeostasis, and disease by combining genetic information, transcriptome, and protein analysis methods based on research on stem cell differentiation processes in various controlled environments. Further, if organoids are produced using human stem cells of a patient, they may be used to develop new drugs or treatments tailored to the patient. Accordingly, respiratory organoids such as of lungs and respiratory tracts may be produced by three-dimensional culture technology and various matrices to structurally reproduce respiratory organs more precisely, and the use of bioengineering organoid production systems is expected to expand to various fields of use such as disease research, toxicity screening, and new drug development.

However, three-dimensional cell cultured organoids as simple various cell clusters have difficulty in increasing the maturity level due to a fairly low degree of differentiation. Further, since most organoids currently adopt culture methods that rely entirely on culture medium instead of supplying nutrition through blood vessels, it is difficult to artificially control the maturity level. Moreover, there is a problem that the maturity level is not constant between organoids, and thus the functional similarity to actual human tissue may decrease. Therefore, when organoids are commercialized by replacing animal models, there is a need for a technology capable of selecting mature organoids showing process standardization and reliability.

Accordingly, in order to realize the uniform efficacy of the organoids, there is a need for an organoid selection technology capable of selecting mature organoids from an image using artificial intelligence.

### DETAILED DESCRIPTION OF THE INVENTION

### Technical Problem

In the foregoing background, there is provided an organoid selection device and method capable of selecting mature organoids from an image using an artificial intelligence model.

### Technical Solution

To achieve the foregoing objects, in an aspect, the present embodiments provide an organoid selection device comprising a learning data generation unit collecting image information about an organoid photographed through an optical microscope and generating a plurality of learning data by performing preprocessing based on the collected image information, a model training unit training an artificial intelligence model by inputting the plurality of learning data to at least one pre-trained model and performing transfer learning, and a selection unit selecting a mature organoid from among organoids by inputting the image information about the organoid to the trained artificial intelligence model.

In another aspect, the present embodiments provide an organoid selection method comprising a learning data generation step collecting image information about an organoid photographed through an optical microscope and generating a plurality of learning data by performing preprocessing based on the collected image information, a model training step training an artificial intelligence model by inputting the plurality of learning data to at least one pre-trained model and performing transfer learning, and a selection step selecting a mature organoid from among organoids by inputting the image information about the organoid to the trained artificial intelligence model.

### BRIEF DESCRIPTION OF DRAWINGS

FIG. 1 is a view illustrating a configuration of an organoid selection device according to an embodiment of the disclosure;

FIG. 2 is a view illustrating an example of image information for an organoid according to an embodiment of the disclosure;

FIG. 3 is a flowchart illustrating operations of a learning data generation unit in an organoid selection device according to an embodiment of the disclosure;

FIG. 4 is a view illustrating an example of generating a plurality of learning data in an organoid selection device according to an embodiment of the disclosure;

FIG. 5 is a flowchart illustrating operations of a model trainer in an organoid selection device according to an embodiment of the disclosure;

FIG. 6 is a view illustrating an example of training an artificial intelligence model in an organoid selection device according to an embodiment of the disclosure;

FIG. 7 is a view illustrating an example of verifying a model using a visualization algorithm in an organoid selection device according to an embodiment of the disclosure;

FIG. 8 is a flowchart illustrating an organoid selection method according to an embodiment of the disclosure; and

FIG. 9 is a block diagram illustrating an organoid selection device according to an embodiment of the disclosure.

### MODE FOR CARRYING OUT THE INVENTION

The disclosure relates to an organoid selection device and method.

Hereinafter, embodiments of the disclosure are described in detail with reference to the accompanying drawings. In assigning reference numerals to components of each drawing, the same components may be assigned the same numerals even when they are shown on different drawings. When determined to make the subject matter of the disclosure unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the present invention. These denotations are provided merely to distinguish a component from another, and the essence, order, or number of the components are not limited by the denotations.

In describing the positional relationship between components, when two or more components are described as “connected”, “coupled” or “linked”, the two or more components may be directly “connected”, “coupled” or “linked” “, or another component may intervene. Here, the other component may be included in one or more of the two or more components that are “connected”, “coupled” or “linked” to each other.

When such terms as, e.g., “after”, “next to”, “after”, and “before”, are used to describe the temporal flow relationship related to components, operation methods, and fabricating methods, it may include a non-continuous relationship unless the term “immediately” or “directly” is used.

When a component is designated with a value or its corresponding information (e.g., level), the value or the corresponding information may be interpreted as including a tolerance that may arise due to various factors (e.g., process factors, internal or external impacts, or noise).

Hereinafter, embodiments of the disclosure are described in detail with reference to the accompanying drawings.

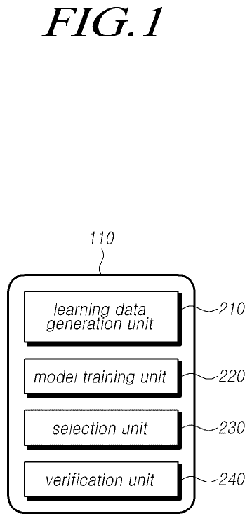

FIG. 1 is a view illustrating a configuration of an organoid selection device according to an embodiment of the disclosure;

Referring to FIG. 1, an organoid selection device 110 according to an embodiment of the disclosure includes a learning data generation unit 210 collecting image information about an organoid photographed through an optical microscope and generating a plurality of learning data by performing preprocessing based on the collected image information, a model training unit 220 training an artificial intelligence model by inputting the plurality of learning data to at least one pre-trained model and performing transfer learning, and a selection unit 230 selecting a mature organoid from among organoids by inputting the image information about the organoid to the trained artificial intelligence model. Further, the organoid selection device 110 according to an embodiment may further include a verification unit 240 for performing verification by dividing the collected image information into a train dataset and a test dataset.

The learning data generation unit 210 according to an embodiment may collect image information about an organoid captured through an optical microscope, and may generate a plurality of learning data by performing preprocessing based on the collected image information. The organoid may be an organoid produced through three-dimensional cell culture from primary cultured cells or stem cells derived from the respiratory system. Specifically, the organoid may be a respiratory tract organoid produced through three-dimensional cell culture from nasal epithelial cells or human nasal turbinate stem cells (hNTSCs).

For example, the learning data generation unit 210 may crop, for each of the collected image information, rectangular image information centered only on the organoid except for an image area not including the organoid. For example, the learning data generation unit 210 may crop out the rectangular image information to include only organoids from an existing size 1920×1080 of the collected image information.

The learning data generation unit 210 may adjust the cropped image information to a size applicable to the artificial intelligence model, and perform normalization by changing the pixel value of the adjusted image information from 0 to 1. For example, the learning data generation unit 210 may adjust the image information cropped into the rectangular shape to a size of 256×256. Further, the learning data generation unit 210 may perform normalization of changing the value between 0 and 255 of the existing pixel value of the adjusted image information to between 0 and 1.

As another example, the learning data generation unit 210 may generate a plurality of learning data by applying at least one of zoom-out, rotation, flip, or contrast adjustment to each of the collected image information. For example, the learning data generation unit 210 may arbitrarily select the collected image information, and may generate new image information by applying at least one of zoom-out, rotation, flip, and contrast adjustment to the selected image information.

As another example, the learning data generation unit 210 may generate a plurality of learning data by associating image information about the organoid with each biomarker. For example, the learning data generation unit 210 may generate a plurality of learning data by labeling the differentiation-related biomarkers with a reference value. Specifically, the learning data generation unit 210 may generate a plurality of learning data by labeling the image information classified for each biomarker with the pcr value related to the maturity level of the organoid. Alternatively, the learning data generation unit 210 may generate a plurality of learning data by labeling the image information about the organoid with information directly determined by an expert in relation to maturity level. The labeled information may be information determined by one or more other inspection methods other than the pcr value. Accordingly, the generated learning data may include labeling information about whether image information for each organoid is mature.

The model training unit 220 according to an embodiment may input the plurality of learning data to at least one pre-trained model and perform transfer learning to train the artificial intelligence model. For example, the learning data generation unit 210 may perform at least one pre-trained model among VGG19, ResNet50, DenseNet121, and EfficientNetB5. The learning data generation unit 210 may train the artificial intelligence model by performing fine-tuning based on the selected pre-trained model. Accordingly, the artificial intelligence model may robustly perform learning at a high learning speed despite a small amount of image information about organoids.

The selection unit 230 according to an embodiment may select a mature organoid by inputting image information about the organoid to the trained artificial intelligence model. For example, the selection unit 230 may select image information about the mature organoid by inputting the new image information to the training-completed artificial intelligence model. For example, the selection unit 230 may recognize a pattern from the image information and select an organoid differentiated at a specific maturity level by the training-completed artificial intelligence model.

The verification unit 240 according to an embodiment may perform verification by classifying the collected image information. For example, the verification unit 240 may perform verification by dividing the collected image information into a train dataset used for training of the artificial intelligence model and a test dataset used to identify the performance of the artificial intelligence model trained through the train dataset. Further, the verification unit 240 may re-divide a validation dataset used to verify the artificial intelligence model at a predetermined ratio from the train datasets. The verification unit 240 may perform verification by cross-verify the verification data set so that the verification data included in the verification data set does not overlap.

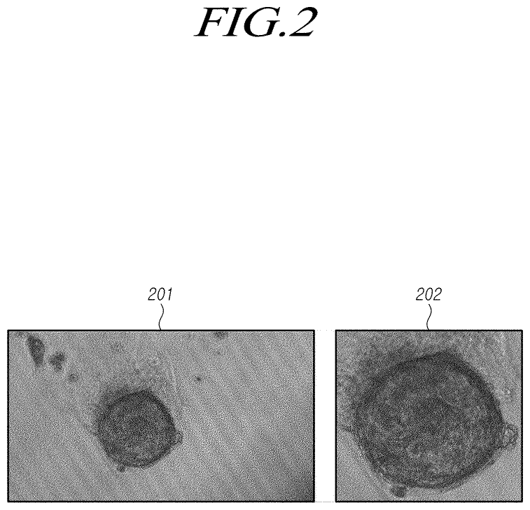

FIG. 2 is a view illustrating an example of image information for an organoid according to an embodiment of the disclosure.

Referring to FIG. 2, image information about the organoid used by the organoid selection device 110 as learning data may be described. For example, organoids may be produced to form specific human tissues through 3D cell culture from primary cultured cells or stem cells derived from the respiratory tract. For example, the primary cultured cells derived from the respiratory tract may be normal cells obtained directly as primary cultured cells directly from the respiratory tissue or organ of the human body. Here, the respiratory tissue may be a paraplegic tissue, but is not limited thereto. Further, the stem cells may include embryonic stem cells, adult stem cells, and induced pluripotent stem cells. Specifically, organoids are three-dimensional cell cultured organoids as simple various cell clusters, and may be respiratory tract organoids produced from nasal epithelial cells or human nasal turbinate stem cells (hNTSCs). Since most organoids currently adopt culture methods that rely entirely on culture medium instead of supplying nutrition through blood vessels, it may be difficult to artificially control the maturity level. In other words, since the maturity level between organoids is not consistent, the functional similarity to actual human tissue may decrease. Accordingly, the organoid selection device 110 may generate an artificial intelligence model for selecting mature organoids showing process standardization and reliability in terms of practical use of organoids.

For example, the organoid selection device 110 may perform training on the image information 201 about the organoid photographed through the optical microscope using the artificial intelligence model, and select the mature organoid. For example, the image information 201 about the organoid may be image information photographed through an optical microscope while culturing the actual organoid. In particular, if the optical microscope is implemented as an automated system, image information about the organoid may be photographed as thousands of images depending on the photographing area and time. Further, the respiratory tract organoid may be cultured after mixing epithelial cells separated from human nasal paraplegic tissue with 60% matrigel. For example, the image information about the respiratory tract organoid may be image information about a single cell that is changed in the spheroid shape photographed through an optical microscope. Specifically, the image information about the respiratory tract organoid may be image information photographed while lumen is formed from a part around 14 days after bronchospheres are formed from a single cell. Accordingly, the learning data generation unit 210 of the organoid selection device 110 may collect the image information 201 about the organoid and perform preprocessing to generate a plurality of learning data.

Further, for example, the learning data generation unit 210 of the organoid selection device 110 may crop, for each of the image information, rectangular image information centered only on the organoid except for the image area not including the organoid. As the image information 201 about the organoid is photographed by an optical microscope, an unwanted image area such as a plate for organoid differentiation or an impurity other than the organoid may also be included in the image information. Accordingly, the learning data generation unit 210 of the organoid selection device 110 may crop rectangular image information 202 including only the organoid from the image information 201 about the organoid.

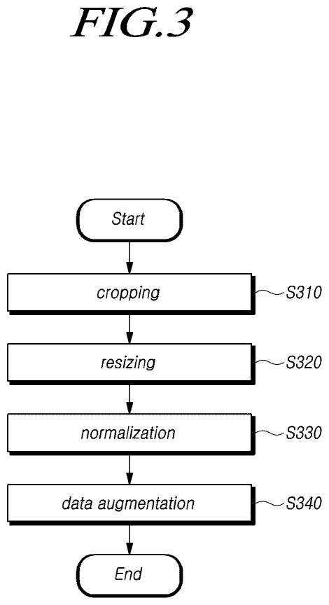

FIG. 3 is a flowchart illustrating operations of a learning data generation unit in an organoid selection device according to an embodiment of the disclosure.

Referring to FIG. 3, the learning data generation unit 210 of the organoid selection device 110 according to an embodiment of the disclosure may crop the collected image information into rectangular image information (S310). For example, the learning data generation unit 210 may crop rectangular image information centered only on the organoid except for the image area not including the organoid, using the image information about the organoid. For example, the learning data generation unit 210 may crop out the rectangular shape from the existing size of 920×1080 of the image information about the organoid, so that only the organoid is centered. Further, the learning data generation unit 210 may exclude low-quality or low-resolution image information due to defocusing.

The learning data generation unit 210 of the organoid selection device 110 according to an embodiment may adjust the cropped image information to a size applicable to the artificial intelligence model (S320). For example, the learning data generation unit 210 may convert the cropped image information into an arbitrarily designated size to adjust to fit the input size of the artificial intelligence model. The arbitrarily designated size is 256×256, which may be a size applicable to the artificial intelligence model. Accordingly, it is possible to prevent the time and memory for training the artificial intelligence model from increasing exponentially.

The learning data generation unit 210 of the organoid selection device 110 according to an embodiment may change and normalize the pixel value of the resized image information (S330). For example, the learning data generation unit 210 may perform normalization by changing the pixel value of the resized image information to between 0 and 1. For example, the learning data generation unit 210 may limit the range of the learning data by performing normalization of changing a value between 0 and 255 of the existing pixel value of the image information to between 0 and 1. Specifically, the learning data generation unit 210 may perform normalizing by calculating an average of existing pixel values, subtracting each pixel value by the average, and dividing it by the standard deviation value. Alternatively, the learning data generation unit 210 may perform normalization by reducing each pixel value by a minimum value among the pixel values and calculating a value obtained by dividing it by the difference between the maximum value and the minimum value among the pixel values. Accordingly, by limiting the data of each dimension to have a value within the same range, it is possible to perform training faster and reduce the possibility of falling into a local optimal state.

The learning data generation unit 210 of the organoid selection device 110 according to an embodiment may augment data based on the collected image information (S330). As an example, the learning data generation unit 210 may generate a plurality of learning data by applying at least one of zoom-out, rotation, flip, or contrast adjustment to each of the collected image information. Data augmentation may be a technique of generating a plurality of learning data through various methods from previously collected image information in order to train an artificial intelligence model using various and large amounts of data. For example, the learning data generation unit 210 may apply zoom-out to secure as wide an area as possible from the organoid included in the collected image information. The learning data generation unit 210 may rotate the collected image information clockwise or counterclockwise. The learning data generation unit 210 may invert the collected image information in a vertical direction or invert the collected image information in an up-down direction and a left-right direction. Further, the learning data generation unit 210 may adjust the color of the collected image information. Specifically, the learning data generation unit 210 may adjust the average RGB value of the collected image information based on the extracted color or randomly. Accordingly, the learning data generation unit 210 may amplify the image information about the organoid to increase the number of data by applying at least one of zoom-out, rotation, flip, and contrast adjustment.

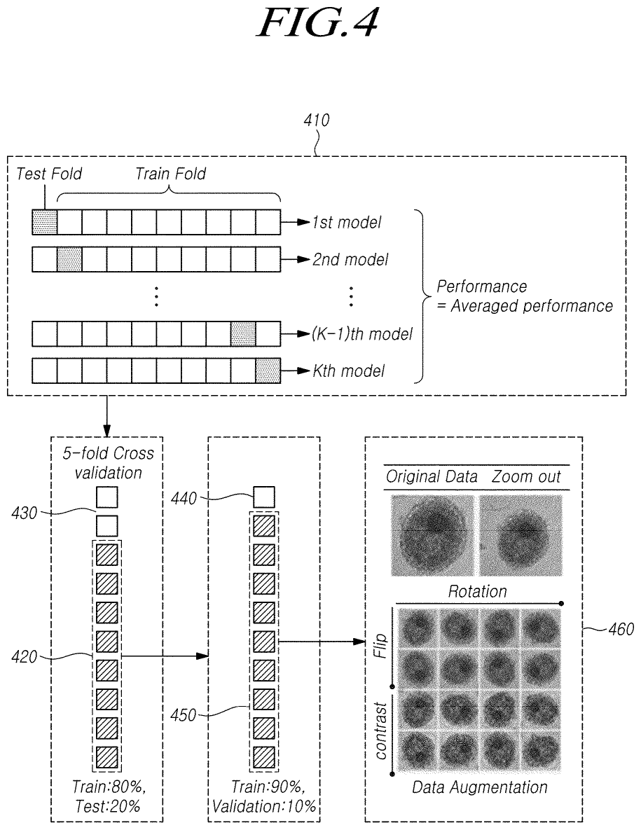

FIG. 4 is a view illustrating an example of generating a plurality of learning data in an organoid selection device according to an embodiment of the disclosure.

Referring to FIG. 4, a plurality of learning data generated by the organoid selection device 110 to train an artificial intelligence model may be described. For example, the collected image information 410 may be randomly divided into a train data set 420 used for training an artificial intelligence model and a test data set 430 used to identify the performance of the artificial intelligence model trained through the train data set. The train data set 420 may be re-divided into a final train data set 450 used for training the artificial intelligence model at a predetermined ratio and a verification data set 440 used to verify the artificial intelligence model. Here, the verification data set 440 may be randomly selected.

Accordingly, the learning data generation unit 210 may generate a plurality of learning data 460 by data-augmenting only image information included in the final train data set 450 except for the test data set 430 and the verification data set 440 among the collected image information 410. Further, the excluded verification data set 440 may be used to perform verification by the verification unit 240 without being used to build and train an artificial intelligence model. In other words, the image information included in the verification data set 440 is new image information that is not used for learning and may be suitable for verification of the artificial intelligence model.

For example, the verification unit 240 may divide the collected image information 410 into the train data set 420 and the test data set 430 to perform cross-verification. For example, the verification unit 240 may perform 5-fold cross-verification using 80% of the collected image information 410 as the train data set 420 and 20% as the test data set 430. Accordingly, substantially the whole image information may be used as the train data set 420 to calculate the final average value as performance, thereby overcoming difficult verification due to the small number of image information. Further, the verification unit 240 may use only 90% of the 80% train data set 420 as the final train data set 450, and the remaining 10% as the verification data set 440. However, the 5-fold cross-validation is described as an example, and the disclosure is not limited thereto. Specifically, when 5-fold cross-verification is performed, the verification unit 240 may change the train data set 420, the test data set 430, and the verification data set 440 five times, and thus output five prediction values of the artificial intelligence model based on the same pre-trained model. The average value of the five prediction values may be used as the final performance of the artificial intelligence model generated based on one pre-trained model.

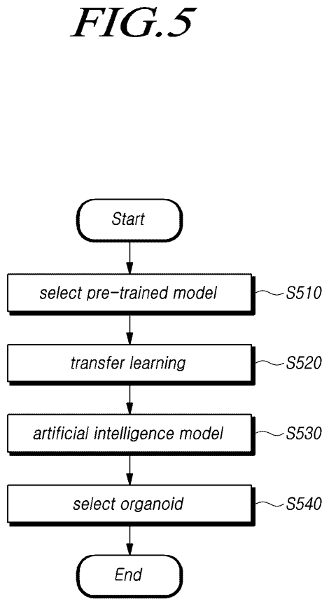

FIG. 5 is a flowchart illustrating operations of a model trainer in an organoid selection device according to an embodiment of the disclosure.

Referring to FIG. 5, the model training unit 220 of the organoid selection device according to an embodiment of the disclosure may select at least one pre-trained model (S510). For example, the model training unit 220 may select at least one pre-trained model from among VGG19, ResNet50, DenseNet121, and EfficientNetB5. Here, the pre-trained model may be a convolutional neural network (CNN)-based deep learning model that extracts a feature of image information and recognizes an image based on the extracted feature. The pre-trained model may be a model having a structure of a general convolutional neural network such as VGG19, ResNet50, DenseNet121, or EfficientNetB5. However, the pre-trained model is not limited thereto as long as it is a model trained with sufficiently large amounts of good quality image information.

The model training unit 220 of the organoid selection device according to an embodiment may perform transfer learning based on the pre-trained model (S520). For example, the model training unit 220 may perform transfer learning to select a mature organoid from image information about the organoid using the selected pre-trained model. For example, the model training unit 220 may retrain the model by performing fine-tuning based on the weight of the training-completed pre-trained model. Here, the fine-tuning may be a technique of initializing the weight to the pre-trained model trained with a large-scale data set (e.g., the image net) including various images in advance, and slightly adjusting the corresponding weight to a small-scale data set. However, fine-tuning is an example of a transfer learning technique, and the disclosure is not limited thereto.

The model training unit 220 of the organoid selection device according to an embodiment may generate an artificial intelligence model through transfer learning (S530). For example, the model training unit 220 may generate an artificial intelligence model that outputs a selection result of a mature organoid using image information about the organoid as an input based on the pre-trained model. For example, the generated artificial intelligence model may be a machine learning model generated through transfer learning from a pre-trained model to select a mature organoid from image information about the organoid.

The selection unit 230 of the organoid selection device according to an embodiment may select the organoid using the training-completed artificial intelligence model (S540). For example, the selection unit 230 may select image information including a mature organoid from the image information about the organoid using the training-completed artificial intelligence model. Here, the mature organoid may be an organoid determined to show uniform efficacy by comparing differentiation results and functions as functional differences occur according to maturity levels. In other words, the mature organoid may be an organoid differentiated with a specific maturity level enough to reproduce a function similar to that of the human body. For example, the selection unit 230 may select the mature organoid by predicting the degree of differentiation of the organoid from the image information about the respiratory tract organoids using the training-completed artificial intelligence model. Further, the selection unit 230 may select the mature organoid by predicting the real-time PCR expression of each biomarker (e.g., MUC5AC, Foxj1, P63, E-cadherin, etc.) using the training-completed artificial intelligence model from the image information.

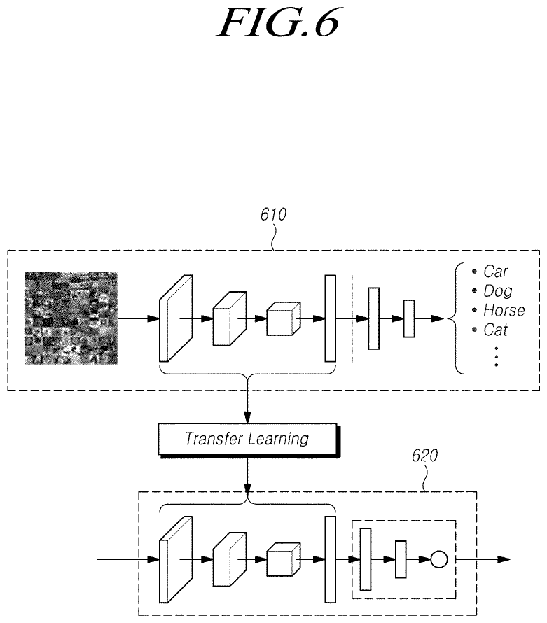

FIG. 6 is a view illustrating an example of training an artificial intelligence model in an organoid selection device according to an embodiment of the disclosure.

Referring to FIG. 6, the model training unit 220 of the organoid selection device 110 according to an embodiment of the disclosure may generate an artificial intelligence model that outputs a selection result of a mature organoid using a plurality of learning data as inputs. For example, the model training unit 220 may additionally insert a fully connected layer modified based on the image information about the organoid into the previously trained pre-trained model 610 and connect nodes, generating the artificial intelligence model 620 for selecting the organoid. The pre-trained model is a deep learning model having a structure of a general convolutional neural network such as VGG19, ResNet50, DenseNet121, EfficientNetB5, or the like, and may include a convolutional layer, a pooling layer, and a fully connected layer. The fully connected layer is also referred to as a dense layer, and may include a weight that connects inputs and outputs all and connects inputs and outputs, respectively. Thus, the convolutional layer and the pooling layer may extract features of the image information, and the fully connected layer may classify the image information into classes. For example, the model training unit 220 may replace the fully connected layer included in the pre-trained model 610 with a new fully connected layer to generate an artificial intelligence model 620 that accurately selects the organoids differentiated with a specific maturity level from the image information about the organoids. The new fully connected layer may be classified for each biomarker, and the output node may be determined according to the biomarker.

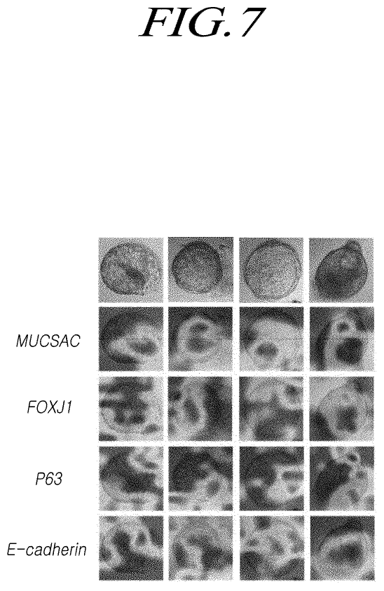

FIG. 7 is a view illustrating an example of verifying a model using a visualization algorithm in an organoid selection device according to an embodiment of the disclosure.

Referring to FIG. 7, the verification unit 240 of the organoid selection device according to an embodiment may display a portion having an important influence on an organoid screening result using at least one algorithm among a plurality of visualization algorithms. Accordingly, the reliability of the artificial intelligence model may be enhanced. For example, the verification unit 240 may additionally verify whether each biomarker is appropriately predicted by displaying the selection result of the artificial intelligence model by applying the visualization algorithm. Here, MUC5AC, Foxj1, P63, and E-cadherin may be used as the biomarker, but the biomarker is not limited thereto. Specifically, MUC5AC may be a biomarker related to mucus secretion expressed in human airway epithelial cells to mucus secretion cells. Foxj1 may be a biomarker that plays an important role in the ciliogenic transcription factor. airway. P63 may be a biomarker of stem cells of various epithelial tissues and basal cells present in precursor cells. Further, E-cadherin may be a biomarker for maintaining epithelial cell structure at the cell boundary.

Further, e.g., the verification unit 240 may analyze spatial importance using gradient-weighted class activation mapping (Grad-CAM) for visualizing the artificial intelligence model using gradient information. Specifically, the verification unit 240 may produce gradient information through backpropagation using the grad-CAM algorithm and obtain a localization map that marks a critical area in the image using the gradient information going to the final layer. Here, the localization map may be obtained with the nonlinear function ReLU after linearly combining the feature map from the final layer and the weight of the gradient. Accordingly, the verification unit 240 may display the portion that has a critical influence on the classification result on the image using the feature map of the final layer. The verification unit 240 may determine the portion having a significant effect on the prediction result by displaying a color according to the calculated relevance score of each pixel.

As another example, the verification unit 240 may evaluate the organoid selection result of the artificial intelligence model generated based on at least one pre-trained model. Specifically, the verification unit 240 may evaluate the organoid selection result as a quantitative result of each artificial intelligence model trained on each biomarker. The respiratory tract organoid screening results of the artificial intelligence model may be evaluated as shown in Table 1.

TABLE 1

| | Correlation by Deep Learning | | | |
|---|---|---|---|---|
| Bio-Marker | VGG19 | ResNet50 | DenseNet121 | EfficientNetB5 |
| FOXJ1 | 0.576 ± 0.165 | 0.880 ± 0.084 | 0.840 ± 0.138 | 0.647 ± 0.382 |
| MUC5AC | 0.062 ± 0.292 | 0.528 ± 0.148 | 0.656 ± 0.163 | 0.226 ± 0.305 |

Hereinafter, an organoid selection method that may be performed by the organoid sorting device described with reference to FIGS. 1 to 7 is described.

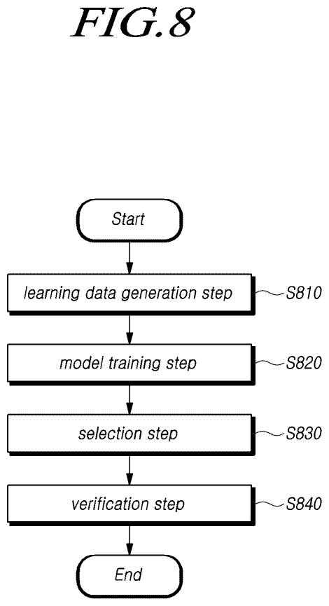

FIG. 8 is a flowchart illustrating an organoid selection method according to another embodiment of the disclosure.

Referring to FIG. 8, an organoid selection method according to an embodiment may include a learning data generation step for generating a plurality of learning data by collecting image information about an organoid (S810). For example, the organoid selection device may collect image information about an organoid captured through an optical microscope, and may generate a plurality of learning data by performing preprocessing based on the collected image information. The organoid may be an organoid produced through three-dimensional cell culture from nasal epithelial cells or human nasal turbinate stem cells (hNTSCs). Specifically, the organoid may be a respiratory tract organoid produced through three-dimensional cell culture from nasal epithelial cells or human nasal turbinate stem cells (hNTSCs).

For example, the organoid selection device may crop, for each of the collected image information, rectangular image information centered only on the organoid except for an image area not including the organoid.

As another example, the organoid selection device may adjust the cropped image information to a size applicable to the artificial intelligence model, and perform normalization by changing the pixel value of the adjusted image information from 0 to 1. Further, the organoid selection device may generate a plurality of learning data by applying at least one of zoom-out, rotation, flip, or contrast adjustment to each of the collected image information.

The organoid selection method according to an embodiment may include a model training step of training the artificial intelligence model based on a pre-trained model (S820). For example, the organoid selection device may input the plurality of learning data to at least one pre-trained model and perform transfer learning to train the artificial intelligence model. For example, the organoid selection device may perform at least one pre-trained model among VGG19, ResNet50, DenseNet121, and EfficientNetB5. The organoid selection device may train the artificial intelligence model by performing fine-tuning based on the selected pre-trained model.

The organoid selection method according to an embodiment may include a selection step of selecting a mature organoid based on the artificial intelligence model (S830). For example, the organoid selection device may select a mature organoid by inputting image information about the organoid to the trained artificial intelligence model. For example, the organoid selection device may select image information about the mature organoid by inputting the new image information to the training-completed artificial intelligence model.

The organoid selection method according to an embodiment may include a verification step of performing verification (S840). For example, the organoid selection device may perform verification by dividing the collected image information into a train dataset used for training of the artificial intelligence model and a test dataset used to identify the performance of the artificial intelligence model trained through the train dataset. For example, the organoid selection device may re-divide a validation dataset used to verify the artificial intelligence model at a predetermined ratio from the train datasets. Further, the organoid selection device may perform verification by cross-verify the verification data set so that the verification data included in the verification data set does not overlap.

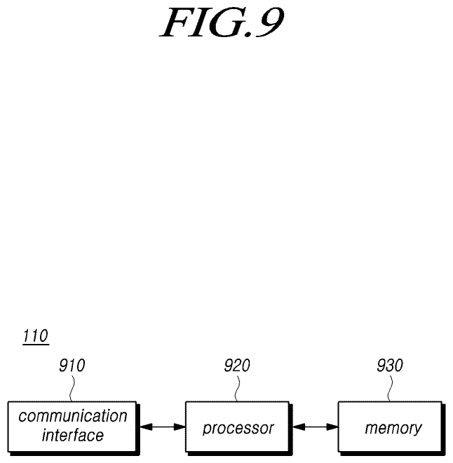

FIG. 9 is a block diagram illustrating an organoid selection device according to an embodiment.

Referring to FIG. 9, an organoid selection device 110 according to an embodiment includes a communication interface 910 and a processor 920. The organoid selection device 110 may further include a memory 930. Each component, communication interface 910, processor 920, and memory 930 may be connected to each other through a communication bus. For example, the communication bus may include a circuit for connecting the components with one another and transferring communications (e.g., control messages and/or data) between the components.

The communication interface 910 may obtain image information about the organoid photographed through an optical microscope. Further, the communication interface 1110 may perform communication with an external device through wireless communication or wired communication.

The processor 920 may perform at least one method described above in connection with FIGS. 1 to 8 or an algorithm corresponding to at least one method. The processor 920 may be a data processing device implemented in hardware having a circuit having a physical structure for executing desired operations. For example, the desired operations may include code or instructions included in a program. For example, the data processing unit implemented in hardware may include a microprocessor, a central processing unit, a processor core, a multi-core processor, a multiprocessor, a neural processing unit (NPU), an application-specific integrated circuit (ASIC), or a field programmable gate array (FPGA).

Further, the processor 920 may execute the program and may control the organoid selection device 110. The program code executed by the processor 920 may be stored in the memory 930.

Information about the pre-trained model and artificial intelligence model including a neural network according to an embodiment of the disclosure may be stored in an internal memory of the processor 920 or may be stored in an external memory, that is, the memory 930. For example, the memory 930 may store image information about the organoid obtained through the communication interface 910. The memory 930 may store an artificial intelligence model including a neural network. Further, the memory 930 may store various information generated during processing by the processor 920 and output information extracted by the processor 920. The output information may be a computation result of the artificial intelligence model or a test result of the artificial intelligence model. The memory 930 may store the learning result of the artificial intelligence model. The learning result of the artificial intelligence model may be obtained from the cell selection device 110 or may be obtained from an external device. The learning result of the artificial intelligence model may include weight and bias values. Further, the memory 930 may store various data and programs. The memory 930 may include a volatile memory or a non-volatile memory. The memory 930 may include a mass storage medium, such as a hard disk and the like, and may store various data.

Although it is described above that all of the components are combined into one or are operated in combination, embodiments of the disclosure are not limited thereto. One or more of the components may be selectively combined and operated as long as it falls within the scope of the objects of the disclosure. Further, although all of the components may be implemented in their respective independent hardware components, all or some of the components may be selectively combined to be implemented in a computer program with program modules performing all or some of the functions combined in one or more hardware components and recorded in a computer-readable medium. The computer-readable medium may include programming commands, data files, or data structures, alone or in combinations thereof. The programming commands recorded in the medium may be specially designed and configured for the present invention or may be known and available to one of ordinary skill in the computer software-related art. Examples of the computer readable recording medium may include, but is not limited to, magnetic media, such as hard disks, floppy disks or magnetic tapes, optical media, such as CD-ROMs or DVDs, magneto-optical media, such as floptical disks, memories, such as ROMs, RAMs, or flash memories, or other hardware devices specially configured to retain and execute programming commands. Examples of the programming commands may include, but are not limited to, high-level language codes executable by a computer using, e.g., an interpreter, as well as machine language codes as created by a compiler. The above-described hardware devices may be configured to operate as one or more software modules to perform operations according to an embodiment of the present invention, or the software modules may be configured to operate as one or more hardware modules to perform the operations.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the present invention should be construed by the following claims, and all technical spirits within equivalents thereof should be interpreted to belong to the scope of the present invention.

### CROSS-REFERENCE TO RELATED APPLICATION

The instant patent application claims priority under 35 U.S.C. 119 (a) to Korean Patent Application No. 10-2022-0022545, filed on Feb. 21, 2022, in the Korean Intellectual Property Office, the disclosure of which is herein incorporated by reference in its entirety. The present patent application claims priority to other applications to be filed in other countries, the disclosures of which are also incorporated by reference herein in their entireties.

1. An organoid selection device, comprising:
a learning data generation unit collecting image information about an organoid photographed through an optical microscope and generating a plurality of learning data by performing preprocessing based on the collected image information;
a model training unit training an artificial intelligence model by inputting the plurality of learning data to at least one pre-trained model and performing transfer learning; and
a selection unit selecting a mature organoid by inputting the image information about the organoid to the trained artificial intelligence model.

2. The organoid selection device of claim 1, wherein the organoid is an organoid produced through three-dimensional cell culture from a primary cultured cell or a stem cell derived from a respiratory system.

3. The organoid selection device of claim 1, wherein the learning data generation unit crops, for each of the collected image information, rectangular image information centered only on the organoid except for an image area not including the organoid.

4. The organoid selection device of claim 3, wherein the learning data generation unit adjusts the cropped image information to a size applicable to the artificial intelligence model, and perform normalization by changing a pixel value of the adjusted image information to between 0 and 1.

5. The organoid selection device of claim 1, wherein the learning data generation unit generates the plurality of learning data by applying at least one of zoom-out, rotation, flip, or contrast adjustment to each of the collected image information.

6. The organoid selection device of claim 1, wherein the model learning unit trains the artificial intelligence model by selecting at least one pre-trained model from among VGG19, ResNet50, DenseNet121, and EfficientNetB5 and performing fine-tuning based on the selected pre-trained model.

7. The organoid selection device of claim 1, further comprising a verification unit performing verification by dividing the collected image information into a train dataset used for training the artificial intelligence model and a test dataset used to identify a performance of the artificial intelligence model trained through the train dataset.

8. The organoid selection device of claim 7, wherein the verification unit re-divides a verification data set used to verify the artificial intelligence model at a preset ratio from the train data set and performs cross-verification on the verification data set so that verification data included in the verification data set does not overlap.

9. An organoid selection method, comprising:
a learning data generation step collecting image information about an organoid photographed through an optical microscope and generating a plurality of learning data by performing preprocessing based on the collected image information;
a model training step training an artificial intelligence model by inputting the plurality of learning data to at least one pre-trained model and performing transfer learning; and
a selection step selecting a mature organoid by inputting the image information about the organoid to the trained artificial intelligence model.

10. The organoid selection method of claim 9, wherein the organoid is an organoid produced through three-dimensional cell culture from a primary cultured cell or a stem cell derived from a respiratory system.

11. The organoid selection method of claim 9, wherein the learning data generation step crops, for each of the collected image information, rectangular image information centered only on the organoid except for an image area not including the organoid.

12. The organoid selection method of claim 11, wherein the learning data generation step adjusts the cropped image information to a size applicable to the artificial intelligence model, and perform normalization by changing a pixel value of the adjusted image information to between 0 and 1.

13. The organoid selection method of claim 9, wherein the learning data generation step generates the plurality of learning data by applying at least one of zoom-out, rotation, flip, or contrast adjustment to each of the collected image information.

14. The organoid selection method of claim 9, wherein the model learning step trains the artificial intelligence model by selecting at least one pre-trained model from among VGG19, ResNet50, DenseNet121, and EfficientNetB5 and performing fine-tuning based on the selected pre-trained model.

15. The organoid selection method of claim 9, further comprising a verification step performing verification by dividing the collected image information into a train dataset used for training the artificial intelligence model and a test dataset used to identify a performance of the artificial intelligence model trained through the train dataset.

16. The organoid selection method of claim 15, wherein the verification step re-divides a verification data set used to verify the artificial intelligence model at a preset ratio from the train data set and performs cross-verification on the verification data set so that verification data included in the verification data set does not overlap.
